(19) United States
(12) Patent Application Publication
(10) Pub. No.: US 2026/0020744 A1
(43) Pub. Date: Jan. 22, 2026

(54) IMAGE GENERATION METHOD FOR MACHINE LEARNING, MACHINE LEARNING METHOD, AND ENDOSCOPE IMAGE PROCESSING APPARATUS

(71) Applicant: OLYMPUS MEDICAL SYSTEMS CORP., Tokyo (JP)

(72) Inventors: Masamitsu HARADA, Tokyo (JP); Sunao KIKUCHI, Tokyo (JP); Tetsuhiro OKA, Tokyo (JP); Yuki NAMII, Tokyo (JP)

(73) Assignee: OLYMPUS MEDICAL SYSTEMS CORP., Tokyo (JP)

(21) Appl. No.: 19/340,267

(22) Filed: Sep. 25, 2025

Related U.S. Application Data
(63) Continuation of application No. PCT/JP2023/013495, filed on Mar. 31, 2023.

(51) Int. Cl. A61B 1/00; G06T 3/4046; G06T 5/20; G06T 5/60; G16H 30/40

(52) U.S. Cl. CPC A61B 1/000096; G06T 3/4046; G06T 5/20; G06T 5/60; G16H 30/40; G06T 2207/10068; G06T 2207/20081; G06T 2207/20084

(57) ABSTRACT

An image generation apparatus for machine learning performs reduction processing on components in at least part of a frequency band higher than a Nyquist frequency of a training image, for a candidate correct answer image with a resolution higher than the training image, and generate a correct answer image. The correct answer image and the training image are used as a pair for machine learning to improve a resolving power of an input image.

### CROSS REFERENCE TO RELATED APPLICATION

This application is a continuation application of PCT/JP2023/013495 filed on Mar. 31, 2023, the entire contents of which are incorporated herein by this reference.

### FIELD

The present disclosure relates to an image generation method for machine learning that generates a correct answer image from a candidate correct answer image with a higher resolution than a training image, a machine learning method that causes a machine learning model to perform learning using the training image and the correct answer image, and an endoscope image processing apparatus that uses the machine learning model that has performed learning.

### BACKGROUND

There are technologies to improve a resolving power of an image, such as a super-resolution technology and an edge emphasis technology. The super-resolution technology increases the number of pixels constituting an image, thereby increasing a resolution compared to the original image. The edge emphasis technology emphasizes edges by increasing specific spatial frequency components in an image.

The technology of performing super-resolution using a machine learning model has been proposed in the past. The machine learning model performs inference on an input image to create an inferred image with an increased resolution, as an output image.

For example, a machine learning model performs super-resolution machine learning using learning data including a pair of a training image and a correct answer image with a higher resolution than the training image. The machine learning model takes the training image as input, performs inference on the training image, and creates an output image. Then, the machine learning model compares the output image with the correct answer image and performs learning to adjust parameters so that a difference between the output image and the correct answer image is reduced. An inference performance of the machine learning model depends on a type of a model to be used and learning data used for machine learning.

For example, Japanese Patent Application Laid-Open Publication No. 2022-70035 recites a method of creating a machine learning model with improved robustness against noises by creating learning data using an image obtained by subjecting a medical input image to noise reduction, as a correct answer image, and an image obtained by downscaling the correct answer image to a low resolution, as a training image.

### SUMMARY

An image generation method for machine learning according to one aspect of the present disclosure includes: for a candidate correct answer image with a resolution higher than a resolution of a training image, the training image being a pair with a correct answer image, performing reduction processing on components in at least part of a frequency band higher than a Nyquist frequency of the training image; and generating the correct answer image for machine learning to improve a resolving power of an input image.

A machine learning method according to one aspect of the present disclosure includes: causing a machine learning model for performing inference to improve a resolving power of an input image and generating an output image to perform learning, using: a correct answer image that is generated by performing reduction processing on components in at least part of a frequency band higher than a Nyquist frequency of a training image, for a candidate correct answer image with a resolution higher than a resolution of the training image, the training image being a pair with the correct answer image, or the correct answer image that is picked up by an image pickup apparatus mounting an optical low-pass filter, the optical low-pass filter being configured to reduce components in at least part of the frequency band higher than the Nyquist frequency of the training image; and the training image.

An endoscope image processing apparatus according to one aspect of the present disclosure includes: a machine learning model connection section that is configured to be connectable to a machine learning model that has performed learning by a machine learning method, the machine learning method causing the machine learning model to perform learning, using: a correct answer image that is generated by performing reduction processing on components in at least part of a frequency band higher than a Nyquist frequency of a training image, for a candidate correct answer image with a resolution higher than a resolution of the training image, the training image being a pair with the correct answer image, or the correct answer image that is picked up by an image pickup apparatus mounting an optical low-pass filter, the optical low-pass filter being configured to reduce components in at least part of the frequency band higher than the Nyquist frequency of the training image; and the training image; and one or more processors. The one or more processors are configured to: input an endoscopic image, which is received, into the machine learning model configured to perform inference to improve a resolving power of an input image and generate an output image; and cause the machine learning model to output the endoscopic image with a resolving power that is improved.

### BRIEF DESCRIPTION OF THE DRAWINGS

FIG. 1 is according to each embodiment of the present disclosure and is a diagram showing a general flow when a machine learning model performs learning for improving a resolving power of an image, thereby becoming a learned model, and performs inference;

FIG. 2 is according to each embodiment and is a diagram showing an example in which the machine learning model is configured by a neural network;

FIG. 3 is according to each embodiment and is a chart showing an example of frequency components of a training image and a correct answer image;

FIG. 4 is according to each embodiment and is a diagram showing an example in which the machine learning model performs learning using the correct answer image generated from a candidate correct answer image;

FIG. 5 is according to each embodiment and is a chart showing first to fourth examples of frequency characteristics of the correct answer image generated from the candidate correct answer image;

FIG. 6 is according to each embodiment and is a graph showing a fifth example of the frequency characteristics of the correct answer image generated from the candidate correct answer image;

FIG. 7 is according to each embodiment and is a graph showing a sixth example of the frequency characteristics of the correct answer image generated from the candidate correct answer image;

FIG. 8 is a block diagram showing a configuration example of an image generation apparatus for machine learning that generates a correct answer image by image processing in a first embodiment of the present disclosure;

FIG. 9 is a block diagram showing a configuration example of an image generation apparatus for machine learning that generates a correct answer image using an optical low-pass filter in a second embodiment of the present disclosure;

FIG. 10 is a block diagram showing a first configuration example of a machine learning apparatus in a third embodiment of the present disclosure;

FIG. 11 is a block diagram showing a second configuration example of a machine learning apparatus in a fourth embodiment of the present disclosure;

FIG. 12 is a block diagram showing a third configuration example of a machine learning apparatus in a fifth embodiment of the present disclosure;

FIG. 13 is a block diagram showing a fourth configuration example of a machine learning apparatus in a sixth embodiment of the present disclosure;

FIG. 14 is a block diagram showing a fifth configuration example of a machine learning apparatus in a seventh embodiment of the present disclosure;

FIG. 15 is a block diagram showing a configuration example of a machine learning apparatus that acquires an original image from an endoscope system in an eighth embodiment of the present disclosure; and

FIG. 16 is a block diagram showing a configuration example in which a learned model that has performed learning in a machine learning apparatus is applied to an endoscope system in a ninth embodiment of the present disclosure.

### DESCRIPTION OF EMBODIMENTS

Hereinafter, embodiments of the present invention will be described with reference to the drawings. However, the present invention is not limited by the embodiments described below.

Note that in the description of the drawings, like or corresponding elements are denoted with the same reference signs as appropriate. It should be noted that the drawings are schematic, and the relationships between the lengths of the various elements, the ratios of the lengths of the various elements, and the quantities of the various elements within a single drawing may differ from reality in order to simplify the description. Furthermore, there may be parts where the relationships and ratios of the lengths of the various elements differ between the drawings.

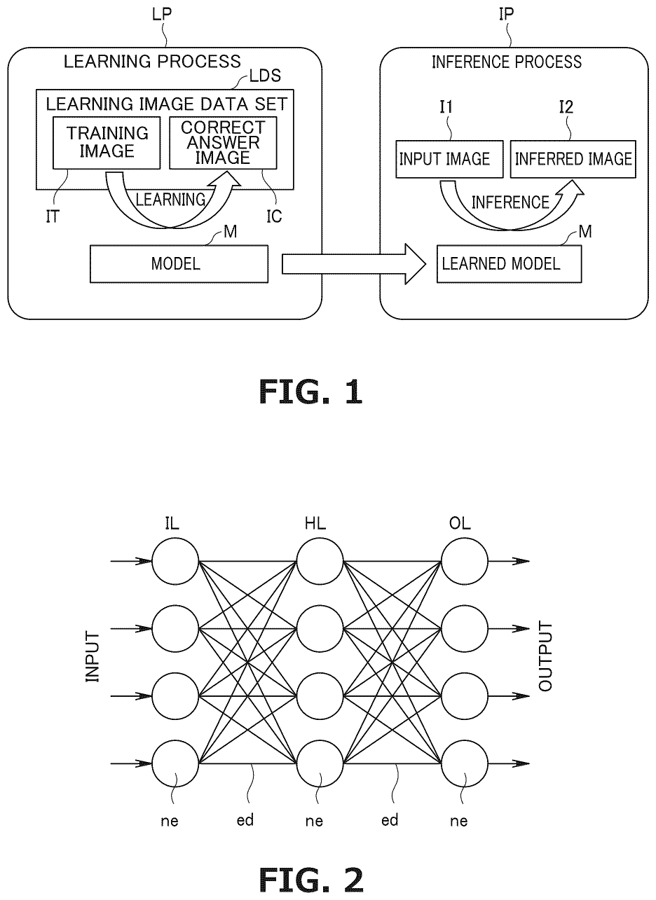

FIG. 1 to FIG. 16 show embodiments of the present disclosure. FIG. 1 is according to each embodiment and is a diagram showing a general flow when a machine learning model M performs learning for improving a resolving power of an image by a machine learning method, thereby becoming a learned model M, and performs inference. Note that in the specification and the drawings, for the sake of simplicity, “machine learning model” is sometimes referred to simply as “model”.

The machine learning model M is, for example, a mathematical model such as a neural network (NN). FIG. 2 is according to each embodiment and is a diagram showing an example in which the machine learning model M is configured by a neural network.

The neural network includes an input layer IL, a hidden layer HL, and an output layer OL. Each of the layers includes a plurality of neurons (units/nodes) ne, and each neuron ne of a certain layer and each neuron ne of the next layer thereof are connected to by edges (lines) ed to form a network structure.

As the machine learning model M, a deep neural network (DNN) with a plurality of hidden layers HL and a total of four or more layers may be used. As the machine learning model M, a convolutional neural network (CNN), Regions with CNN features (R-CNN) or Fully Convolutional Networks (FCN) that use the CNN, or the like, may be used. Note that machine learning is not limited to deep learning, and other various well-known learning methods may also be used.

The machine learning model M performs machine learning using learning data LDS in a learning process LP, to which the machine learning method is applied. The learning data LDS is a learning image data set including a pair of a training image IT and a correct answer image IC. The learning data LDS including the correct answer image IC is also referred to as teacher data. The machine learning model M takes the training image IT as input, and forwardly propagates data of the inputted training image IT in the order of the input layer IL, the hidden layer HL, and the output layer OL to generate an output image.

The machine learning model M then performs learning by calculating a difference (loss value) between the output image and the correct answer image IC using a loss function and, in a backward propagation, adjusting parameters to make the loss value as small as possible using an optimization algorithm.

The machine learning model M that has performed learning through the learning process LP performs inference on the input image I1 in an inference process IP, and an inferred image I2 is the output image. The machine learning model M, which has performed learning, is configured by a combination of an Artificial Intelligence (AI) program (algorithm) and the parameters optimized through learning, for example.

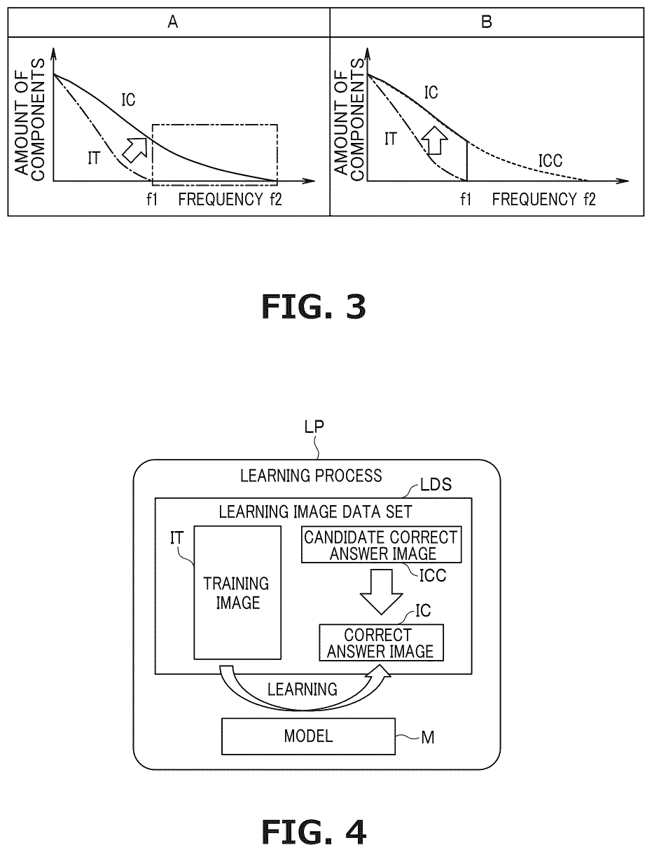

FIG. 3 is according to each embodiment and is a chart showing an example of frequency components of the training image IT and the correct answer image IC. Note that in the specification, a frequency refers to a spatial frequency.

Section A of FIG. 3 shows an example of a graph of the frequency components of the training image IT and the correct answer image IC when the correct answer image IC with a higher resolution than the training image IT is used. When the correct answer image IC has a higher resolution than the training image IT, the correct answer image IC generally includes components (high frequency components) in a higher frequency band (higher than f1 and equal to or lower than f2) than a frequency band (equal to or higher than 0 and equal to or lower than f1) of the training image IT, as shown in a part surrounded by the long dashed double-short dashed line in section A of FIG. 3. Here, f1 is an upper limit frequency at which the training image IT includes components, and f2 is an upper frequency at which the correct answer image IC includes components.

If a combination of the training image IT and the correct answer image IC as shown in section A of FIG. 3 is used, the machine learning model M performs learning to generate an output image in the frequency band equal to or higher than 0 and equal to or lower than f2. However, since the high frequency components in the frequency band higher than f1 and equal to or lower than f2 are information that does not originally exist in the training image IT, the inference results by the learned model are not guaranteed. As a result, the learned model may perform a false inference to generate a false pattern, and in medical images, for example, generate an image that appears to have blood vessels in a position where there are originally no blood vessels.

Section B of FIG. 3 shows an example of a graph of frequency components of the training image IT, the candidate correct answer image ICC, and the correct answer image IC when the correct answer image IC is generated from the candidate correct answer image ICC with a higher resolution than the training image IT.

The candidate correct answer image ICC shown in section B of FIG. 3 has the same frequency characteristics as the correct answer image IC shown in section A of FIG. 3. The correct answer image IC is generated by reducing components of the candidate correct answer image ICC on the higher frequency side than the frequency f1, without reducing components, equal to or lower than the frequency f1, of the candidate correct answer image ICC. Here, “reducing” means that reducing the amount of the components of the correct answer image IC to less than the amount of the components of the candidate correct answer image ICC at the same frequency, and also includes making the amount of the components 0.

FIG. 4 is according to each embodiment and is a diagram showing an example in which the machine learning model M performs learning using the correct answer image IC generated from the candidate correct answer image ICC.

In the learning process LP, the machine learning model M uses, for learning, the training image IT, and the correct answer image IC generated by reducing the components on the higher frequency side than the frequency f1 from the candidate correct answer image ICC. This can reduce the false inferences in the frequency bands where components of the training image IT do not originally exist, when inference is performed using the learned model M. For example, the false inferences can be suppressed in which an image that appears to have blood vessels in the position where there are no blood vessels is generated.

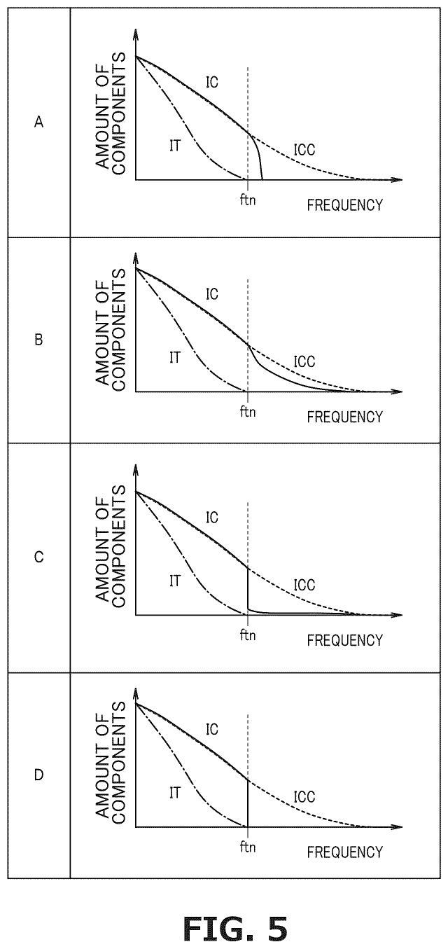

FIG. 5 is according to each embodiment and is a chart showing first to fourth examples of the frequency characteristics of the correct answer image IC generated from the candidate correct answer image ICC.

In relation to each section of FIG. 5, the training image IT is composed of a plurality of pixels arranged at a first pixel pitch, and the first pixel pitch is a sampling distance. A sampling frequency of the training image IT is a reciprocal of the sampling distance. A Nyquist frequency ftn of the training image IT is ½ of the value of the sampling frequency. The training image IT includes components in the frequency band equal to or lower than the Nyquist frequency ftn, and does not include components in the frequency band higher than the Nyquist frequency ftn (referred to as the high frequency band as appropriate).

In addition, the candidate correct answer image ICC is composed of a plurality of pixels arranged at a second pixel pitch that is smaller than the first pixel pitch, and the second pixel pitch is the sampling distance. Therefore, the candidate correct answer image ICC generally includes components up to a frequency band higher than the Nyquist frequency ftn of the training image IT.

Furthermore, none of the correct answer images IC shown in each section of FIG. 5 change in the amount of the components in the frequency band equal to or lower than the Nyquist frequency ftn from the amount of the components of the candidate correct answer image ICC.

The correct answer image IC of the first example shown in section A of FIG. 5 has frequency characteristics in which the amount of the components of the candidate correct answer image ICC is rapidly and smoothly reduced when the frequency exceeds the Nyquist frequency ftn (becomes higher than the Nyquist frequency ftn), and the amount of the components is made 0 at a certain frequency higher than the Nyquist frequency ftn.

The correct answer image IC of the second example shown in section B of FIG. 5 has frequency characteristics in which the amount of the components of the candidate correct answer image ICC in the high frequency band is reduced overall. Therefore, the correct answer image IC includes the components also in the high frequency band, but the amount of the components of the correct answer image IC in the high frequency band is smaller than the amount of the components of the candidate correct answer image ICC.

For the correct answer image IC of the third example shown in section C of FIG. 5, the amount of the components of the candidate correct answer image ICC is discontinuously reduced to a value close to 0 when the frequency exceeds the Nyquist frequency ftn (becomes higher than the Nyquist frequency ftn). The correct answer image IC has frequency characteristics with the amount of the components close to 0 in the high frequency band.

For the correct answer image IC of the fourth example shown in section D of FIG. 5, the amount of the components is discontinuously made 0 when the frequency exceeds the Nyquist frequency ftn (becomes higher than the Nyquist frequency ftn), and the amount of the components is made 0 in the overall high frequency band.

Incidentally, the resolution of the image includes a relative resolution, a value of which changes depending on the size of a paper surface of printed matter or the size of a display screen, on which the image is to be displayed, or the like (in other words, depending on the number of pixels per unit length), and an absolute resolution, which is determined by the number of pixels that constitutes the image. In the specification, the resolution refers to the absolute resolution.

To take one numerical example, assume that the candidate correct answer image ICC is composed of 1280 horizontal by 720 vertical pixels, and the training image IT is composed of 640 horizontal by 360 vertical pixels.

The correct answer images IC shown in sections A to C of FIG. 5 include components in the frequency band higher than the Nyquist frequency ftn. Therefore, in the above numerical example, the correct answer image IC is composed of 1280 horizontal by 720 vertical pixels, similar to the candidate correct answer image ICC, for example.

On the other hand, the correct answer image IC shown in section D of FIG. 5 does not include components in the frequency band higher than the Nyquist frequency ftn. Therefore, in the numerical example described above, the correct answer image IC may be composed of 1280 horizontal by 720 vertical pixels similar to the candidate correct answer image ICC, but may be composed of 640 horizontal by 360 vertical pixels similar to the training image IT. In the latter case, downscaling processing may be performed on the candidate correct answer image ICC to generate the correct answer image IC.

As above, the correct answer image IC does not need to have the resolution higher than the training image IT, as with the candidate correct answer image ICC, and may have the same resolution as the training image IT.

Note that the correct answer image IC shown in section A of FIG. 5 includes the components up to a frequency slightly higher than the Nyquist frequency ftn, but the frequency band on the high frequency side is narrower than that of the candidate correct answer image ICC. Therefore, in the numerical example described above, the correct answer image IC may have a pixel composition between 640 horizontal by vertical 360 pixels and 1280 horizontal by 720 vertical pixels. Also in this case, the downscaling processing may be performed on the candidate correct answer image ICC to generate the correct answer image IC.

The relationship between the resolution of the correct answer image IC and the resolution of the training image IT is as described above, and the relationship of resolving powers is as follows.

When the amount of the components of the correct answer image IC and the amount of the components of the training image IT are compared in a frequency band equal to or lower than the Nyquist frequency ftn, the amount of components of the correct answer image IC is more than that of the training image IT. In particular, on the high frequency side in the frequency band equal to or lower than the Nyquist frequency ftn, the amount of the components of the correct answer image IC is more than that of the training image IT.

Therefore, even if the resolutions of the correct answer image IC and the training image IT are the same, the resolving power of the correct answer image IC is higher than that of the training image IT. Note that, in general, as the resolving power of an image increases, so does the contrast of the image. Therefore, the contrast of the correct answer image IC is higher than the contrast of the training image IT.

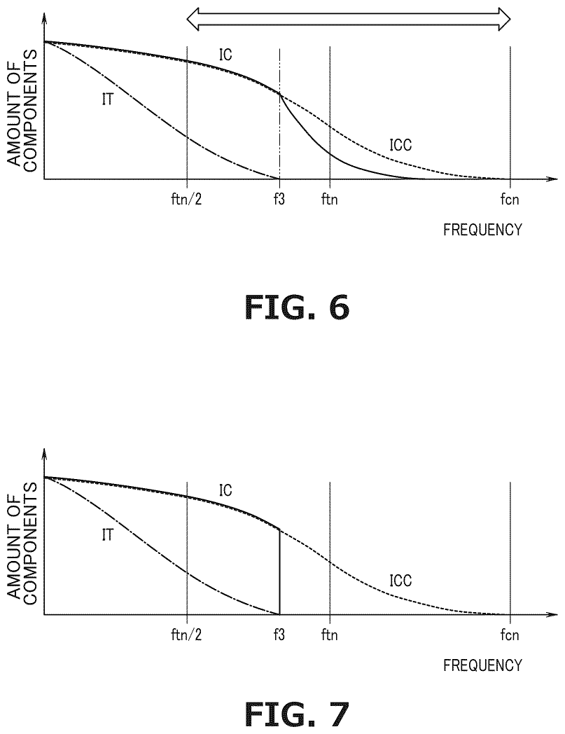

FIG. 6 is according to each embodiment and is a graph showing a fifth example of the frequency characteristics of the correct answer image IC generated from the candidate correct answer image ICC.

In each of the examples shown in FIG. 5, the amount of the components of the correct answer image IC is reduced in the frequency band higher than the Nyquist frequency ftn compared to the candidate correct answer image ICC, but is not reduced in the frequency band equal to or lower than the Nyquist frequency ftn. In contrast, in the fifth example shown in FIG. 6, the amount of the components of the correct answer image IC is reduced also in part of the frequency band equal to or lower than the Nyquist frequency ftn.

The training image IT shown in FIG. 6 does not include components in part of the high frequency side of the frequency band, which is equal to or lower than the Nyquist frequency ftn and equal to or higher than ½ of the Nyquist frequency ftn. In this case, even in the frequency band equal to or lower than the Nyquist frequency ftn, where the training image IT does not include information, faults may occur in the inference by the learned model.

Therefore, in the fifth example shown in FIG. 6, processing is performed to reduce components in at least part of the frequency band (the frequency band indicated by the hollow bidirectional arrow in FIG. 6) equal to or higher than ½ of the Nyquist frequency ftn and equal to or lower than a Nyquist frequency fcn of the candidate correct answer image ICC. In this way, the components may be reduced or removed as necessary also in the frequency band equal to or lower than the Nyquist frequency ftn.

Specifically, in the fifth example shown in FIG. 6, the training image IT includes components in a frequency band equal to or lower than a frequency f3 indicated by the long dashed double-short dashed line, and does not include components in a frequency band higher than the frequency f3. The frequency f3 is a frequency equal to or higher than half of the Nyquist frequency ftn (ftn/2) and lower than the Nyquist frequency ftn.

Therefore, the correct answer image IC is generated by reducing the components of the candidate correct answer image ICC in the frequency band higher than the frequency f3 and equal to or lower than the Nyquist frequency fcn of the candidate correct answer image ICC. The fifth example shown in FIG. 6 shows an example of frequency characteristics of the correct answer image IC in which the amount of components of the candidate correct answer image ICC is reduced overall in the frequency band higher than the frequency f3, in accordance with the second example shown in section B of FIG. 5.

FIG. 7 is according to each embodiment and is a graph showing a sixth example of the frequency characteristics of the correct answer image IC generated from the candidate correct answer image ICC.

The sixth example shown in FIG. 7 shows an example in which frequency characteristics of the correct answer image IC are different from the frequency characteristics of the correct answer image IC in the fifth example shown in FIG. 6, in a case where the frequency characteristics of the training image IT and the candidate correct answer image ICC are the same as the frequency characteristics of the training image IT and the candidate correct answer image ICC in the fifth example shown in FIG. 6.

In other words, the amount of components of the correct answer image IC of the sixth example shown in FIG. 7 is discontinuously made 0 when the frequency exceeds the frequency f3 (becomes higher than the frequency f3), and the amount of the components in the frequency band higher than the frequency f3 is overall 0, in accordance with the fourth example shown in section D of FIG. 5.

As described with reference to FIG. 6 and FIG. 7, the correct answer image IC may be generated by reducing the amount of the components of the candidate correct answer image ICC in the frequency band higher than the frequency f3, which is the upper limit of the frequency band in which the training image IT includes the components. The upper limit frequency f3 can be obtained, for example, by a frequency analysis of the training image IT.

In addition, for example, when 1/2≤α<1, the frequency analysis may be omitted, and the correct answer image IC may be generated by reducing the amount of the components of the candidate correct answer image ICC in a frequency band equal to or higher than α×ftn.

In this case, if α=1/2, the amount of the components of the candidate correct answer image ICC in a frequency band equal to or higher than half of the Nyquist frequency ftn (ftn/2) is reduced to generate the correct answer image IC. If α=1/2, false inferences can be prevented in many practical cases.

### First Embodiment

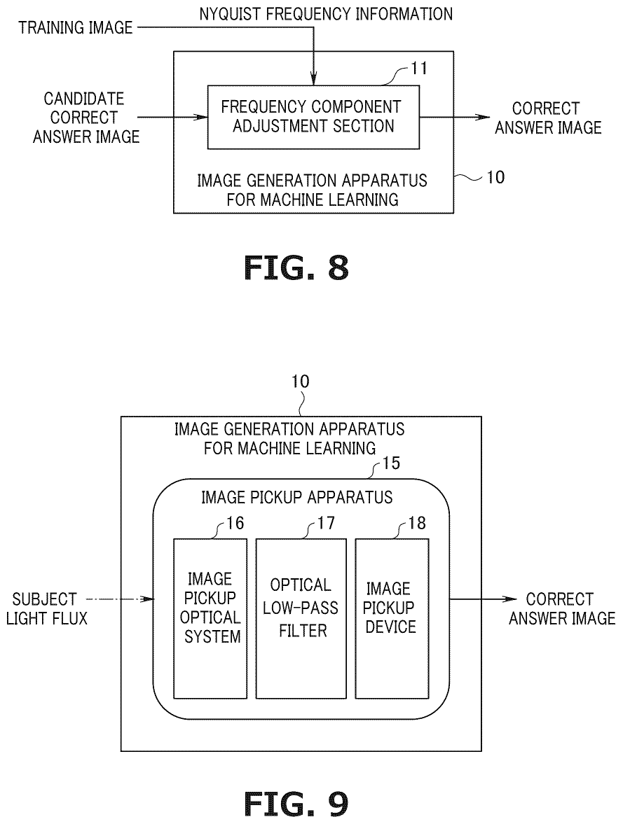

FIG. 8 is a block diagram showing a configuration example of an image generation apparatus for machine learning 10 that generates the correct answer image IC by image processing in a first embodiment. The image generation apparatus for machine learning 10 shown in FIG. 8 generates the correct answer image IC from the candidate correct answer image ICC by an image generation method for machine learning using the image processing.

The image generation apparatus for machine learning 10 includes a frequency component adjustment section 11. The frequency component adjustment section 11 is hardware. The frequency component adjustment section 11 receives information on the Nyquist frequency ftn of the training image IT, and the candidate correct answer image ICC with the higher resolution than the training image IT. The frequency component adjustment section 11 performs reduction processing on the components of the candidate correct answer image ICC in at least part of the frequency band higher than the Nyquist frequency ftn, to generate the correct answer image IC used for machine learning to improve the resolving power of the image.

The reduction processing by the frequency component adjustment section 11 may be processing to reduce the components in the overall frequency band higher than the Nyquist frequency ftn, as shown in section B and section C of FIG. 5.

In addition, the reduction processing by the frequency component adjustment section 11 may be processing to make the components 0 in the overall frequency band higher than the Nyquist frequency ftn (reduce so as to become 0), as shown in section D of FIG. 5. In other words, the reduction processing includes removal processing to make the frequency components 0.

The frequency component adjustment section 11 may perform the reduction processing of the components in the overall frequency band higher than the Nyquist frequency ftn by downscaling the candidate correct answer image ICC. Here, the downscaling processing is to change the pixel composition of the candidate correct answer image ICC to reduce the number of pixels. The correct answer image IC generated by the downscaling processing on the candidate correct answer image ICC has the resolution lower than the resolution of the candidate correct answer image ICC and equal to or higher than the resolution of the training image IT.

In the numerical example described above, in which the candidate correct answer image ICC is composed of 1280 horizontal by 720 vertical pixels and the training image IT is composed of 640 horizontal by 360 vertical pixels, the downscaling processing is processing that makes the correct answer image IC have the pixel composition of 640 or more and less than 1280 horizontal pixels, and 360 or more and less than 720 vertical pixels.

In the downscaling processing, for example, the components of the candidate correct answer image ICC in the frequency band equal to or lower than the Nyquist frequency ftn may not be reduced. With this, in the frequency band equal to or lower than the Nyquist frequency ftn, the resolving power of the correct answer image IC does not deteriorate compared to the resolving power of the candidate correct answer image ICC.

In addition to the processing that does not deteriorate the resolving power of the correct answer image IC compared to the resolving power of the candidate correct answer image ICC, further processing may be performed to improve the resolving power of the correct answer image IC compared to the resolving power of the candidate correct answer image ICC in the frequency band equal to or lower than the Nyquist frequency ftn.

The reduction processing by the frequency component adjustment section 11 may be processing to downscale the candidate correct answer image ICC so that the resolution becomes the same as the resolution of the training image IT.

The reduction processing by the frequency component adjustment section 11 is not limited to the frequency band higher than the Nyquist frequency ftn, as described above. In other words, the reduction processing by the frequency component adjustment section 11 may further include processing to reduce the components in at least part of the frequency band equal to or higher than ½ of the Nyquist frequency ftn and equal to or lower than the Nyquist frequency ftn.

In addition, the reduction processing by the frequency component adjustment section 11 may be performed by applying low-pass filter processing as image processing to the candidate correct answer image ICC.

Alternatively, the reduction processing by the frequency component adjustment section 11 may be performed by both the low-pass filter processing and the downscaling processing. For example, the reduction processing by the frequency component adjustment section 11 may be performed by applying the low-pass filter processing to the candidate correct answer image ICC, and performing the downscaling processing on the candidate correct answer image ICC to which the low-pass filter processing has been applied.

According to the first embodiment, the frequency component adjustment section 11 reduces the components of the candidate correct answer image ICC in the at least part of the frequency band higher than the Nyquist frequency ftn, to generate the correct answer image IC. Therefore, the possibility that the machine learning model, which has performed learning using the training image IT and the correct answer image IC, generates an output image that falsely inferred can be reduced.

In particular, by making the components in the overall frequency band higher than the Nyquist frequency ftn 0, the false inference such as generation of false patterns can be significantly reduced.

In addition, when the reduction processing is performed by at least one of the downscaling processing or the low-pass filter processing, the processing load can be significantly reduced and the processing speed can be improved, compared to, for example, a case where processing of performing Fourier analysis to reduce the components in the frequency band higher than (or equal to or higher than) a specific frequency and then performing inverse Fourier transformation for restoring, is performed.

By not reducing the resolving power of the correct answer image IC less than the resolving power of the candidate correct answer image ICC in the frequency band equal to or lower than the Nyquist frequency ftn, the machine learning model can perform learning to generate the inferred image with a higher resolving power.

### Second Embodiment

FIG. 9 is a block diagram showing a configuration example of an image generation apparatus for machine learning 10 that generates the correct answer image IC using an optical low-pass filter 17 in a second embodiment. In the second embodiment, the same parts as those in the first embodiment are denoted with the same reference signs, and the descriptions thereof are omitted, as appropriate. In the second embodiment, points different from the first embodiment will be mainly described.

In the first embodiment, the correct answer image IC is generated from the candidate correct answer image ICC by the image processing. In contrast, the image generation apparatus for machine learning 10 of the second embodiment shown in FIG. 9 generates the correct answer image IC by an image generation method for machine learning in which an image pickup device 18 picks up an image of a subject light flux that passes through the optical low-pass filter 17.

An image pickup apparatus 15 that generates the correct answer image IC is, for example, an image pickup system including an image pickup optical system 16, the optical low-pass filter 17, and the image pickup device 18. The image pickup optical system 16, the optical low-pass filter 17, and the image pickup device 18 are hardware.

The image pickup optical system 16 collects the subject light flux to form an optical image of a subject on an image pickup surface of the image pickup device 18. The image pickup optical system 16 generally includes a plurality of optical lenses and an optical aperture. However, the image pickup optical system 16 may have other configuration.

The optical low-pass filter 17 is disposed on a passing path of the subject light flux, and cuts high frequency components of the optical image and allows low frequency components to pass. The optical low-pass filter 17 is generally disposed on the image pickup surface of the image pickup device 18. However, the optical low-pass filter 17 may be disposed in another position.

The optical low-pass filter 17 reduces the high frequency components of the optical image in at least part of the frequency band higher than the Nyquist frequency ftn of the training image IT. As a specific example, the optical low-pass filter 17 reduces the frequency components of the optical image in the frequency band equal to or higher than ½ of the Nyquist frequency ftn.

The image pickup device 18 photoelectrically converts the optical image of the subject, which is formed by the image pickup optical system 16 through the optical low-pass filter 17, and outputs signals. An image related to the outputted signals is the correct answer image IC in which the components in the at least part of the frequency band higher than the Nyquist frequency ftn are reduced.

According to the second embodiment, by picking up the optical image of the subject, which is formed through the optical low-pass filter 17, the correct answer image IC capable of reducing the faults in the inference and suitable for learning can be generated. In this case, the image processing that performs the reduction processing on the candidate correct answer image ICC to generate the correct answer image IC, as in the first embodiment, is not required.

Note that the correct answer image IC used by the machine learning model M for learning together with the training image IT may be either the correct answer image IC generated in the configuration of the first embodiment or the correct answer image IC generated in the configuration of the second embodiment.

### Third Embodiment

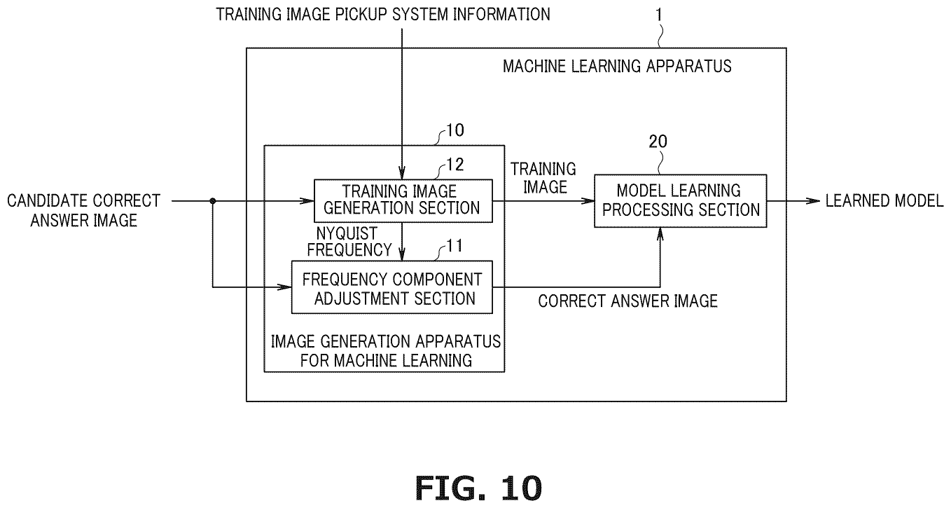

FIG. 10 is a block diagram showing a first configuration example of a machine learning apparatus 1 in a third embodiment. In the third embodiment, the same parts as those in the first and second embodiments are denoted with the same reference signs, and the descriptions thereof are omitted, as appropriate. In the third embodiment, points different from the first and second embodiments will be mainly described.

The machine learning apparatus 1 includes an image generation apparatus for machine learning 10 and a model learning processing section 20. The image generation for machine learning apparatus 10 and the model learning processing section 20 are hardware. The image generation apparatus for machine learning 10 of the present embodiment generates the training image IT and the correct answer image IC from the candidate correct answer image ICC. The model learning processing section 20 performs machine learning using the training image IT and the correct answer image IC that are generated.

The machine learning apparatus 1 may be configured to perform functions of each of the sections by a processor such as an application specific integrated circuit (ASIC) including a central processing unit (CPU), etc. and a field programmable gate array (FPGA) reading and executing a processing program stored in a storage apparatus (or a recording medium) such as a memory. The machine learning apparatus 1 may be configured as a dedicated electronic circuit that performs the functions of each of the sections. The processor, the memory, and the dedicated electronic circuit are hardware.

The image generation apparatus for machine learning 10 includes the frequency component adjustment section 11 and a training image generation section 12. The training image generation section 12 is hardware. The processor or the dedicated electronic circuit may operate as the frequency component adjustment section 11, the training image generation section 12, or the model learning processing section 20. The frequency component adjustment section 11 and the training image generation section 12 receive the candidate correct answer image ICC.

The training image generation section 12 further receives training image pickup system information. The training image pickup system information is image pickup system information assumed for the training image IT. Note that in other embodiments described later, image pickup system information assumed for the correct answer image IC (correct answer image pickup system information) is used. Regardless of whether the training image IT or the correct answer image IC is assumed, the image pickup system information includes pixel number information, color information, optical characteristic information, noise characteristic information, color filter information, etc.

The pixel number information is information on the pixel composition of the image, and is information corresponding to the pixel composition of the image pickup device of an endoscope to which the machine learning model M is applied. The pixel number information assumed for the training image IT is information composed of 640 horizontal by 360 vertical pixels in the case of the numerical example described above.

The color information is information for correcting a difference between a color of an input image (the candidate correct answer image ICC in the present embodiment) and a color of an output image (the training image IT in the present embodiment).

The optical characteristic information includes, for example, information (PSF for correction) for correcting a point spread function (PSF). The PSF is a point spread function that indicates how a point light source is formed in a spatial distribution image by the image pickup optical system.

The PSF of the input image (the candidate correct answer image ICC in the present embodiment) is generally different from the PSF assumed for the output image (the training image IT in the present embodiment). Therefore, the optical characteristic information for converting the PSF of the input image to the PSF assumed for the output image is the PSF for correction.

Here, the PSF of the image pickup optical system depends on the position in the image in general. Therefore, a PSF depending on the coordinates in the image may be used as the PSF for correction. In addition, using the PSF for correction that is uniform across the screen has the advantage that a circuit scale and a calculation cost can be reduced.

Note that examples of the PSF assumed for the training image IT include an actual PSF in the image pickup optical system of the endoscope to which the machine learning model M is applied, or a virtual PSF. The virtual PSF may have more blur than the actual PSF. By using the virtual PSF with more blur than the actual PSF, it is expected that an image for which the learned model M performs inference has a greatly improved resolving power. On the other hand, if the virtual PSF with less blur is used, it is expected that the image for which the learned model M performed inference has fewer false inferences although the resolving power deteriorates.

The noise characteristic information is information that indicates how much noise is to be added to a pixel value of which position, assuming random noise, for example. The noise characteristic information may include both information on a noise position and information on a noise amount, but may include only at least one of the information on the noise position, the information on the noise amount, or information on standard deviation of the added noise. When using the information on the standard deviation of the noise as the noise characteristic information, the information on the standard deviation of the noise may be uniform regardless of the pixel value, or may be variable depending on the pixel value.

The color filter information is information related to such as, for example, whether the endoscope to which the machine learning model M is applied is a type of including an image pickup device including a Bayer array color filter or a type of emitting RGB illumination light in a plane sequential method and picking up an image by a monochrome image pick up device to sequentially acquire R, G, B images. Hereinafter, an image acquired by the image pickup device including the Bayer array color filter is referred to as a Bayer image, and an image acquired by the monochrome image pickup device using the plane sequential emitting is referred to as a plane sequential image.

The training image generation section 12 generates the training image IT from the candidate correct answer image ICC based on the training image pickup system information described above.

The training image generation section 12 performs the downscaling processing on the candidate correct answer image ICC, for example, from 1280 horizontal by 720 vertical pixels to 640 horizontal by 360 vertical pixels based on the pixel number information, to generate a first intermediate image.

The training image generation section 12 performs color correction processing on the first intermediate image based on the color information, to generate a second intermediate image.

Furthermore, the training image generation section 12 performs PSF correction by applying the PSF for correction depending on the coordinates in the image to the second intermediate image, to generate a third intermediate image that has the blur corresponding to the PSF assumed for the training image IT.

In addition, the training image generation section 12 adds random noises, etc. to the third intermediate image based on the noise characteristic information assumed for the training image IT, to generate a fourth intermediate image.

If the candidate correct answer image ICC is a Bayer image and the endoscope to which the machine learning model M is applied generates a Bayer image, or if the candidate correct answer image ICC is a plane sequential image and the endoscope to which the machine learning model M is applied generates a plane sequential image, the training image generation section 12 outputs the fourth intermediate image as the training image IT.

On the other hand, if the candidate correct answer image ICC is a plane sequential image and the endoscope to which the machine learning model M is applied generates a Bayer image, the training image generation section 12 converts the fourth intermediate image, which is the plane sequential image, to a Bayer image and outputs the converted fourth intermediate image as the training image IT.

In addition, when the endoscope generates a plane sequential image, it is preferable that the candidate correct answer image ICC is a plane sequential image. However, the training image generation section 12 may perform demosaic processing on the fourth intermediate image generated from the candidate correct answer image ICC, which is the Bayer image, to convert the fourth intermediate image to a plane sequential image, and output the converted fourth intermediate image as the training image IT.

Note that, in the above description, one example of the processing order when the training image generation section 12 generates the training image IT from the candidate correct answer image ICC based on each information included in the training image pickup system information is described, but the processing order is not limited to the above and the processing may be performed in other orders.

The training image generation section 12 transmits the generated training image IT to the model learning processing section 20 and transmits the information on the Nyquist frequency ftn to the frequency component adjustment section 11.

As described above, the frequency component adjustment section 11 receives the information on the Nyquist frequency ftn of the training image IT and the candidate correct answer image ICC with the higher resolution than the training image IT. The frequency component adjustment section 11 reduces the components of the candidate correct answer image ICC in the frequency band higher than the Nyquist frequency ftn (or equal to or higher than ½ of the Nyquist frequency ftn), to generate the correct answer image IC, as in the first embodiment.

The model learning processing section 20 causes the machine learning model M to perform learning by the learning process LP using the training image IT and the correct answer image IC, as described with reference to FIG. 4. The model learning processing section 20 outputs the learned model M.

According to the third embodiment, substantially the same effects as those in the first embodiment are provided. In addition, according to the third embodiment, when the candidate correct answer image ICC is acquired, both the training image IT and the correct answer image IC can be generated.

Furthermore, according to the third embodiment, since the training image generation section 12 corrects the input image using the training image pickup system information, the training image IT that is highly consistent with the correct answer image IC can be generated. The learned model M, which has performed machine learning using the training image IT and the correct answer image IC that are highly consistent with each other, is expected to generate an inferred image with a more appropriately improved resolving power.

### Fourth Embodiment

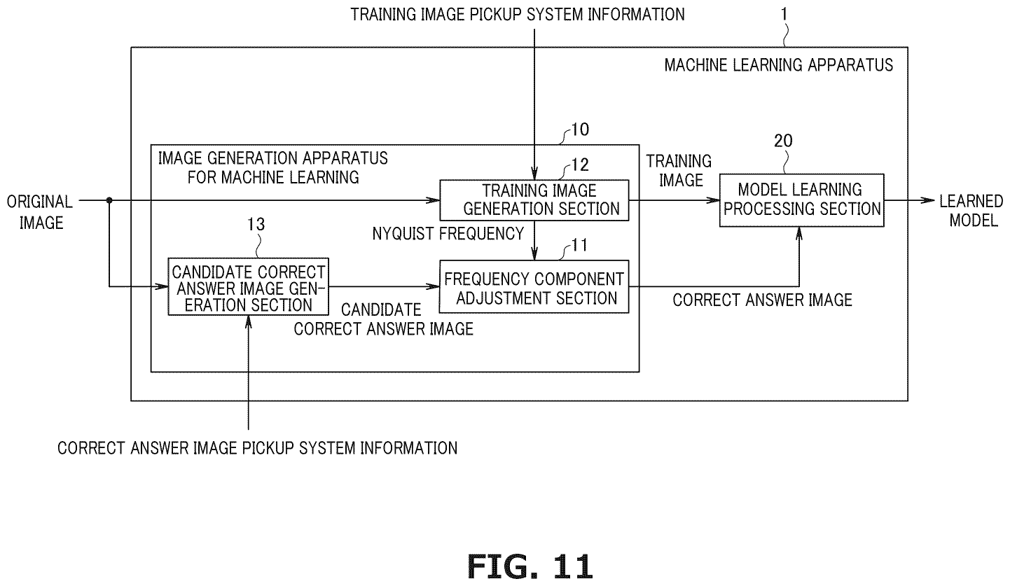

FIG. 11 is a block diagram showing a second configuration example of a machine learning apparatus 1 in a fourth embodiment. In the fourth embodiment, the same parts as those in the first to third embodiments are denoted with the same reference signs, and the descriptions thereof are omitted, as appropriate. In the fourth embodiment, points different from the first to third embodiments will be mainly described.

The machine learning apparatus 1 includes an image generation apparatus for machine learning 10 and a model learning processing section 20. The image generation apparatus for machine learning 10 of the present embodiment generates the training image IT and the correct answer image IC from an original image. The model learning processing section 20 performs machine learning using the training image IT and the correct answer image IC that are generated.

The image generation apparatus for machine learning 10 includes the frequency component adjustment section 11, the training image generation section 12, and a candidate correct answer image generation section 13. The candidate correct answer image generation section 13 is hardware. A processor or a dedicated electronic circuit may operate as the candidate correct answer image generation section 13. The training image generation section 12 and the candidate correct answer image generation section 13 receive the original image.

The training image generation section 12 further receives the training image pickup system information as described above. The training image generation section 12 generates the training image IT from the original image based on the training image pickup system information. The training image generation section 12 transmits the generated training image IT to the model learning processing section 20, and transmits the information on the Nyquist frequency ftn to the frequency component adjustment section 11.

The candidate correct answer image generation section 13 further receives the correct answer image pickup system information. As described above, the correct answer image pickup system information is the image pickup system information assumed for the correct answer image IC. Generally, values of each information included in the correct answer image pickup system information are different from values of each information included in the training image pickup system information. The candidate correct answer image generation section 13 generates the candidate correct answer image ICC from the original image in the same procedure as in the training image generation section 12, based on the correct answer image pickup system information. The candidate correct answer image generation section 13 transmits the generated candidate correct answer image ICC to the frequency component adjustment section 11.

The frequency component adjustment section 11 generates the correct answer image IC from the candidate correct answer image ICC based on the Nyquist frequency ftn, as described above.

The model learning processing section 20 causes the machine learning model M to perform learning using the training image IT and the correct answer image IC, and outputs the learned model M, as described above.

According to the fourth embodiment, substantially the same effects as those in the first and third embodiments are provided. In addition, both the training image IT and the correct answer image IC can be generated from one original image by using the correct answer image pickup system information in addition to the training image pickup system information.

### Fifth Embodiment

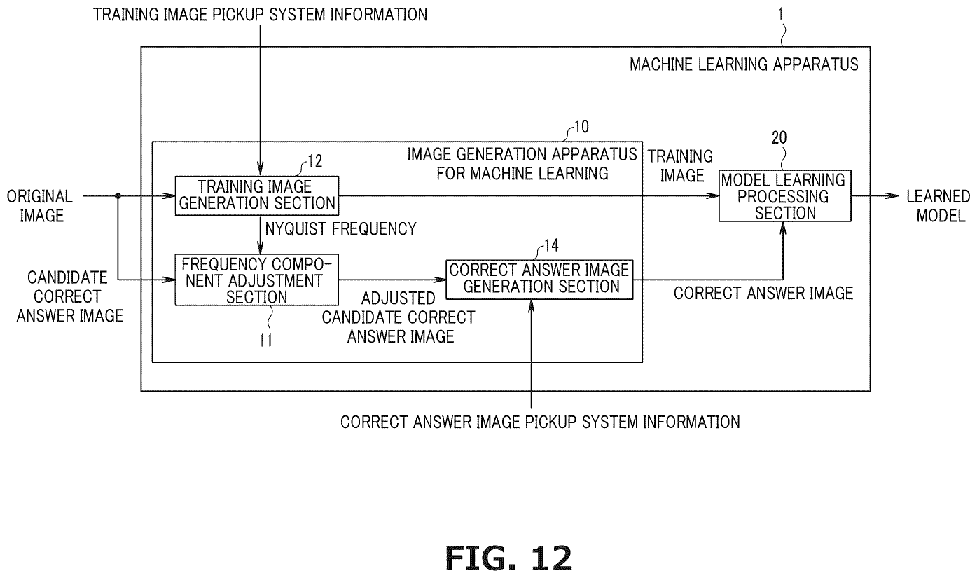

FIG. 12 is a block diagram showing a third configuration example of a machine learning apparatus 1 in a fifth embodiment. In the fifth embodiment, the same parts as those in the first to fourth embodiments are denoted with the same reference signs, and the descriptions thereof are omitted, as appropriate. In the fifth embodiment, points different from the first to fourth embodiments will be mainly described.

An image generation apparatus for machine learning 10 of the present embodiment generates the training image IT and the correct answer image IC from the original image as in the fourth embodiment. However, the processing order by the image generation apparatus for machine learning 10 of the present embodiment is different from that in the fourth embodiment.

The machine learning apparatus 1 includes the image generation apparatus for machine learning 10 and a model learning processing section 20. The image generation apparatus for machine learning 10 includes the frequency component adjustment section 11, the training image generation section 12, and a correct answer image generation section 14. The correct answer image generation section 14 is hardware. A processor or a dedicated electronic circuit may operate as the correct answer image generation section 14. The frequency component adjustment section 11 and the training image generation section 12 receive the original image.

The training image generation section 12 further receives the training image pickup system information. The training image generation section 12 generates the training image IT from the original image based on the training image pickup system information. The training image generation section 12 transmits the generated training image IT to the model learning processing section 20 and transmits the information on the Nyquist frequency ftn to the frequency component adjustment section 11.

The frequency component adjustment section 11 receives the original image as the candidate correct answer image ICC. The frequency component adjustment section 11 reduces the components of the candidate correct answer image ICC in the frequency band higher than the Nyquist frequency ftn (or equal to or higher than 1/2 of the Nyquist frequency ftn) to generate the candidate correct answer image ICC that is adjusted, as with the frequency component adjustment section 11 described in the first embodiment. The frequency component adjustment section 11 transmits the adjusted candidate correct answer image ICC to the correct answer image generation section 14.

The correct answer image generation section 14 receives the adjusted candidate correct answer image ICC. The correct answer image generation section 14 further receives the correct answer image pickup system information. The correct answer image generation section 14 generates the correct answer image IC from the adjusted candidate correct answer image ICC based on the correct answer image pickup system information, in the same procedure as in the candidate correct answer image generation section 13. The correct answer image generation section 14 transmits the generated correct answer image IC to the model learning processing section 20.

The model learning processing section 20 causes the machine learning model M to perform learning using the training image IT and the correct answer image IC, and outputs the learned model M, as described above.

In the fourth embodiment, after performing the image processing on the original image based on the correct answer image pickup system information, the frequency component adjustment section 11 performs the reduction processing. In contrast, in the fifth embodiment, after the frequency component adjustment section 11 performs the reduction processing on the original image, the image processing based on the correct answer image pickup system information is performed to generate the correct answer image IC. According to the fifth embodiment, substantially the same effects as those in the fourth embodiment are provided.

### Sixth Embodiment

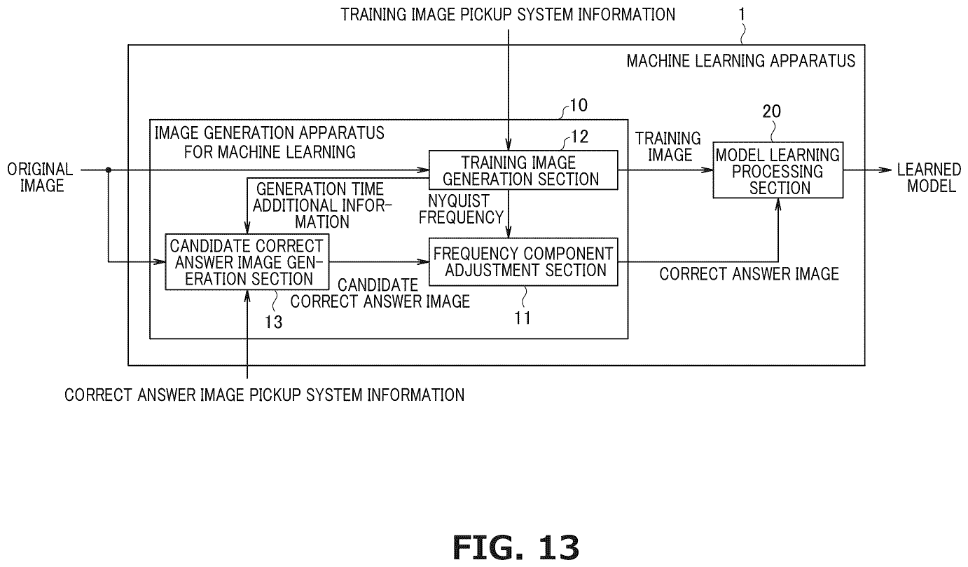

FIG. 13 is a block diagram showing a fourth configuration example of a machine learning apparatus 1 in a sixth embodiment. In the sixth embodiment, the same parts as those in the first to fifth embodiments are denoted with the same reference signs, and the descriptions thereof are omitted, as appropriate. In the sixth embodiment, points different from the first to fifth embodiments will be mainly described.

The machine learning apparatus 1 of the present embodiment includes the same constituent sections as in the fourth embodiment, but information to be transmitted is different from that in the fourth embodiment.

The machine learning apparatus 1 includes an image generation apparatus for machine learning 10 and a model learning processing section 20. The image generation apparatus for machine learning 10 includes the frequency component adjustment section 11, the training image generation section 12, and the candidate correct answer image generation section 13.

The training image generation section 12 generates the training image IT from the original image, transmits the training image IT to the model learning processing section 20, and transmits the information on the Nyquist frequency ftn to the frequency component adjustment section 11, as in the fourth embodiment. The training image generation section 12 of the present embodiment further transmits generation time additional information to the candidate correct answer image generation section 13.

The generation time additional information is information that indicates positions and an amount of noises added to the training image IT by the training image generation section 12, and/or standard deviation information of noises included in the training image pickup system information.

The candidate correct answer image generation section 13 processes the original image based on the correct answer image pickup system information. At this time, the correct answer image pickup system information of the sixth embodiment does not include noise characteristic information. Therefore, the candidate correct answer image generation section 13 adds noises to the processed original image based on the generation time additional information to generate the candidate correct answer image ICC. In addition, if there are a plurality of candidate positions corresponding to the positions of the noises added to the training image IT in the candidate correct answer image ICC, the same noises added to the training image IT may be added to a pixel value of each of the plurality of candidate positions. Alternatively, the same noises added to the training image IT may be added to pixel values of part of the plurality of candidate positions, and random noises newly generated from the standard deviation information of the noises may be added to pixel values of other candidate positions.

The candidate correct answer image generation section 13 then transmits the generated candidate correct answer image ICC to the frequency component adjustment section 11.

The frequency component adjustment section 11 generates the correct answer image IC from the candidate correct answer image ICC based on the Nyquist frequency ftn, as described above.

In the above-described fourth embodiment, the training image IT and the correct answer image IC are each individually added with the noises. In contrast, in the sixth embodiment, the noises added to the training image IT and the correct answer image IC coincide with each other in terms of the positions and the amounts of the noises at corresponding portions.

According to the sixth embodiment, substantially the same effects as those in the fourth embodiment are provided. Furthermore, according to the sixth embodiment, since the positions and the amounts of the noises added to the training image IT and the correct answer image IC coincide with each other, the machine learning model M can perform learning to infer the correct answer image IC from the training image IT, prioritizing the improvement of the resolving power.

### Seventh Embodiment

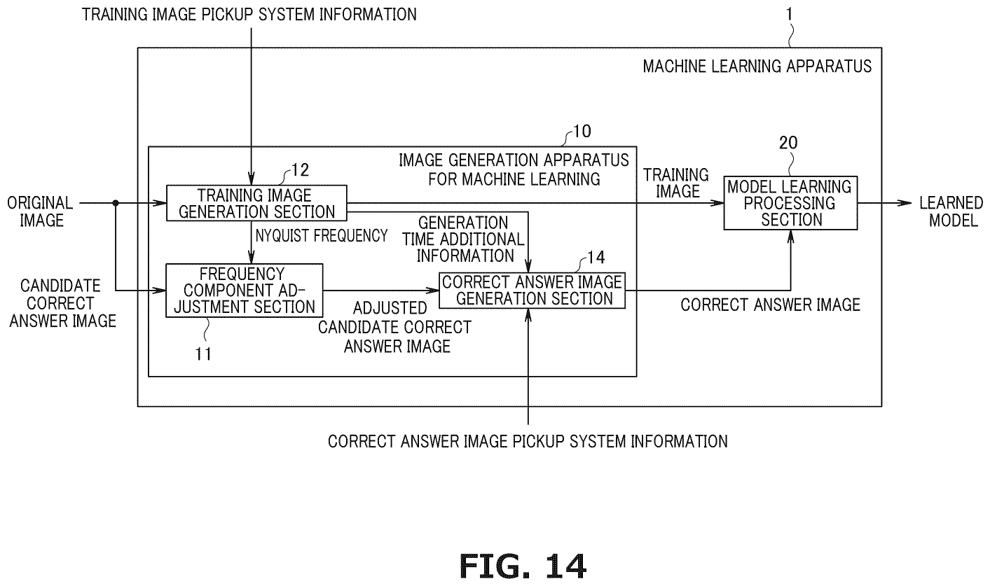

FIG. 14 is a block diagram showing a fifth configuration example of a machine learning apparatus 1 in a seventh embodiment. In the seventh embodiment, the same parts as those in the first to sixth embodiments are denoted with the same reference signs, and the descriptions thereof are omitted, as appropriate. In the seventh embodiment, points different from the first to sixth embodiments will be mainly described.

The machine learning apparatus 1 of the present embodiment includes the same constituent sections as in the fifth embodiment, but information to be transmitted is different from that in the fifth embodiment.

The machine learning apparatus 1 includes an image generation apparatus for machine learning 10 and a model learning processing section 20. The image generation apparatus for machine learning 10 includes the frequency component adjustment section 11, the training image generation section 12, and the correct answer image generation section 14.

The training image generation section 12 generates the training image IT from the original image, transmits the training image IT to the model learning processing section 20, and transmits the information on the Nyquist frequency ftn to the frequency component adjustment section 11, as in the fifth embodiment. The training image generation section 12 of the present embodiment further transmits the generation time additional information described above to the correct answer image generation section 14.

The correct answer image generation section 14 processes the adjusted candidate correct answer image based on the correct answer image pickup system information. At this time, the correct answer image pickup system information of the seventh embodiment does not include the noise characteristic information. The correct answer image generation section 14 then adds the noises to the adjusted candidate correct answer image that is processed, based on the generation time additional information to generate the correct answer image IC.

In the fifth embodiment described above, the training image IT and the correct answer image IC are each individually added with the noises, but in the seventh embodiment, the noises added to the training image IT and the correct answer image IC coincide with each other in terms of the positions and the amounts of the noises at corresponding portions.

After that, the correct answer image generation section 14 transmits the generated correct answer image IC to the model learning processing section 20.

According to the seventh embodiment, substantially the same effects as those in the fifth embodiment are provided. Furthermore, according to the seventh embodiment, since the positions and the amounts of the noises added to the training image IT and the correct answer image IC coincide with each other, the machine learning model M can perform learning to infer the correct answer image IC from the training image IT, prioritizing the improvement of the resolving power.

### Eighth Embodiment

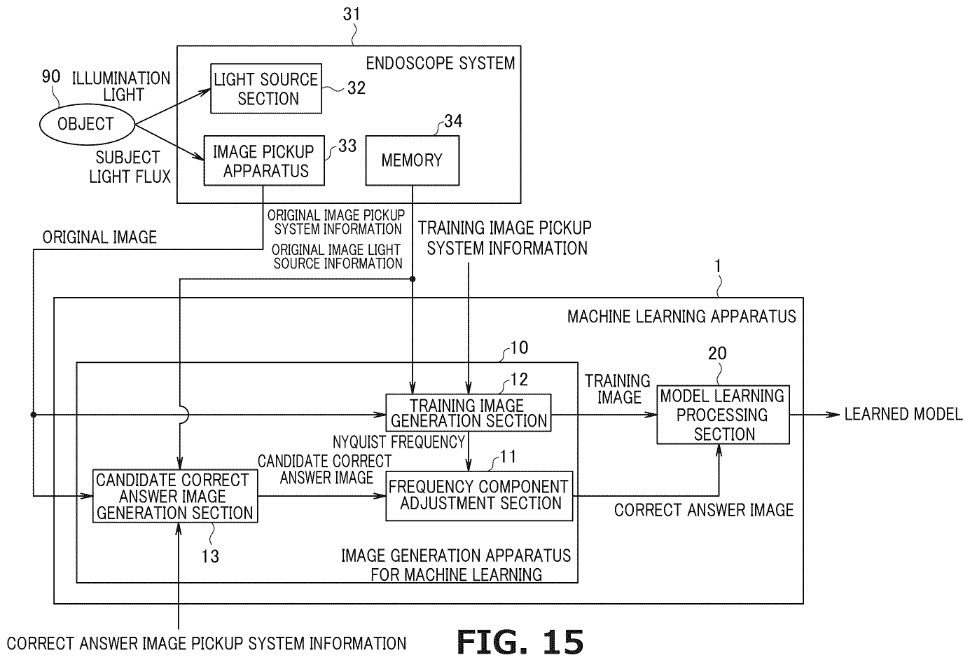

FIG. 15 is a block diagram showing a configuration example of a machine learning apparatus 1 that acquires an original image from an endoscope system 31 in an eighth embodiment. In the eighth embodiment, the same parts as those in the first to seventh embodiments are denoted with the same reference signs, and the descriptions thereof are omitted, as appropriate. In the eighth embodiment, points different from the first to seventh embodiments will be mainly described.

The machine learning apparatus 1 of the present embodiment acquires the original image from the endoscope system 31.

The machine learning apparatus 1 includes an image generation apparatus for machine learning 10 and a model learning processing section 20. The image generation apparatus for machine learning 10 includes the frequency component adjustment section 11, the training image generation section 12, and the candidate correct answer image generation section 13.

Processing of generating the learned model M by the machine learning apparatus 1 is basically the same as the one described with reference to FIG. 11. However, the machine learning apparatus 1 further acquires original image pickup system information and original image light source information from the endoscope system 31 and performs the processing.

Note that FIG. 15 shows the configuration of the machine learning apparatus 1 shown in FIG. 11 as one example, but the configuration of the machine learning apparatus 1 shown in any of FIG. 12 to FIG. 14 may be applied to the present embodiment. In addition, when an image generated by the endoscope system 31 is used as the candidate correct answer image ICC, the configuration of the machine learning apparatus 1 shown in FIG. 10 may be applied to the present embodiment.

The endoscope system 31 includes a light source section 32, an image pickup apparatus 33, and a memory 34. The light source section 32, the image pickup apparatus 33, and the memory 34 are hardware. The light source section 32 irradiates an object 90, which is a subject, with illumination light. A subject light flux, which is return light from the object 90, enters the image pickup apparatus 33.

The image pickup apparatus 33 roughly has a configuration similar to that of the image pickup apparatus 15 shown in FIG. 9, except that the optical low-pass filter 17 is removed. The image pickup apparatus 33 forms an image of the subject light flux by the image pickup optical system 16, picks up an image by the image pickup device 18, and outputs the original image. As described with reference to FIG. 11, the original image is inputted to the training image generation section 12 and the candidate correct answer image generation section 13.

The memory 34 is a storage medium that stores information related to the endoscope system 31 in a non-volatile manner. The information stored in the memory 34 includes the original image pickup system information and the original image light source information. The original image pickup system information is image pickup system information related to the image pickup apparatus 33 that acquires the original image.

The original image pickup system information includes pixel number information of the image pickup device 18, color information of an image acquired by the image pickup apparatus 33, optical characteristic information including the PSF of the image pickup optical system 16, noise characteristic information related to the image pickup device 18 and a reading circuit and the like from the image pickup device 18, color filter information indicating that the image pickup device 18 acquires which of the Bayer image or the plane sequential image, and the like.

The light source section 32 is, for example, configured to be capable of emitting the illumination light corresponding to a plurality kinds of observation modes. The observation modes include, for example, a white light imaging (WLI) mode, a narrow band imaging (NBI) mode, and the like. The original image light source information is information indicating a kind of the illumination light (the WLI illumination light, the NBI illumination light, and the like) emitted by the light source section 32 according to each of the observation modes.

The endoscope system 31 transmits from the memory 34 the original image pickup system information, and the original image light source information according to the observation mode to the machine learning apparatus 1.

The training image generation section 12 and the candidate correct answer image generation section 13 of the machine learning apparatus 1 receive the original image pickup system information and the original image light source information from the endoscope system 31.

The training image generation section 12 changes, for example, the PSF for correction and the noise characteristic information included in the training image pickup system information, according to the kind of the illumination light (the WLI illumination light, the NBI illumination light, and the like), based on the original image light source information. At this time, the training image generation section 12 may further change the PSF for correction and the noise characteristic information included in the training image pickup system information, based on the optical characteristic information and the noise characteristic information included in the original image pickup system information.

Similarly, the candidate correct answer image generation section 13 changes, for example, the PSF for correction and the noise characteristic information included in the correct answer image pickup system information, according to the kind of the illumination light (the WLI illumination light, the NBI illumination light, and the like), based on the original image light source information. At this time, the candidate correct answer image generation section 13 may further change the PSF for correction and the noise characteristic information included in the correct answer image pickup system information, based on the optical characteristic information and the noise characteristic information included in the original image pickup system information.

Note that when the learned model M is applied to the same model (or even the same individual) as the endoscope system 31 that acquires the original image, only addition of the noises may be performed without performing the PSF correction.

In addition, the training image generation section 12 and the candidate correct answer image generation section 13 may change, for example, the downscaling processing, as necessary, based on the pixel number information of the image pickup device 18 included in the original image pickup system information.

Furthermore, the training image generation section 12 and the candidate correct answer image generation section 13 may change, for example, the color correction processing, as necessary, based on the color information included in the original image pickup system information.

The training image generation section 12 and the candidate correct answer image generation section 13 may change, for example, the conversion processing from the Bayer image to the plane sequential image or from the plane sequential image to the Bayer image, as necessary, based on the color filter information included in the original image pickup system information.

According to the eighth embodiment, substantially the same effects as in the third to seventh embodiments can be provided by acquiring the original image from the endoscope system 31. In addition, according to the eighth embodiment, an appropriate learning image data set can be generated by appropriately changing the training image pickup system information and the correct answer image pickup system information based on at least one of the original image pickup system information or the original image light source information.

### Ninth Embodiment

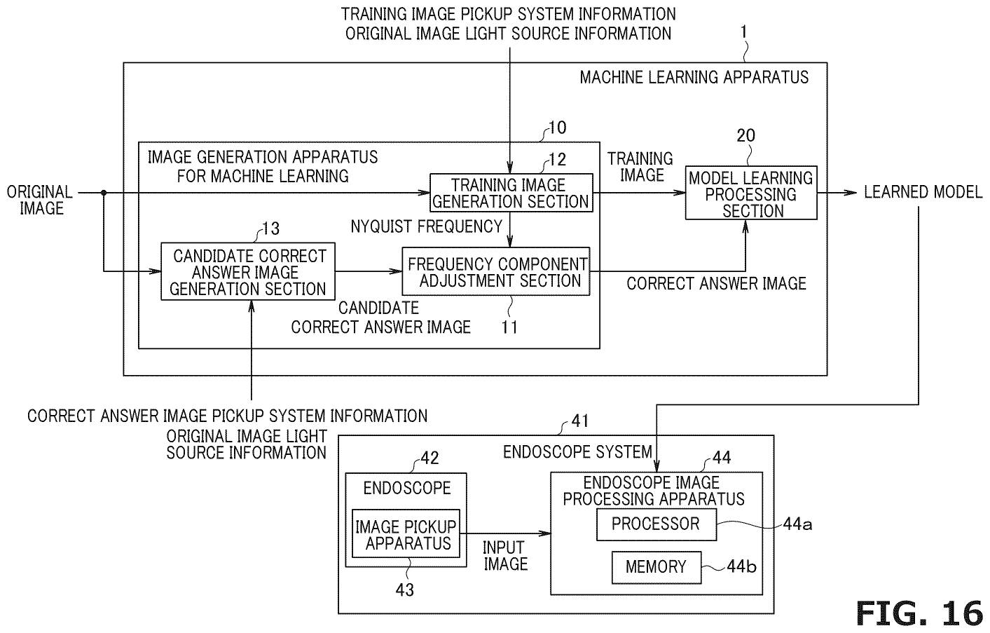

FIG. 16 is a block diagram showing a configuration example in which a learned model M that has performed learning by a machine learning apparatus 1 is applied to an endoscope system 41 in a ninth embodiment. In the ninth embodiment, the same parts as those in the first to eighth embodiments are denoted with the same reference signs, and the descriptions thereof are omitted, as appropriate. In the ninth embodiment, points different from the first to eighth embodiments will be mainly described.

The machine learning apparatus 1 includes an image generation apparatus for machine learning 10 and a model learning processing section 20. The image generation apparatus for machine learning 10 includes the frequency component adjustment section 11, the training image generation section 12, and the candidate correct answer image generation section 13.

The processing of generating the learned model M by the machine learning apparatus 1 is in accordance with the one described with reference to FIG. 15. In other words, in addition to the processing described with reference to FIG. 11, the original image light source information is further acquired and processing is performed.

Note that FIG. 16 shows the configuration of the machine learning apparatus 1 shown in FIG. 11 as one example, but the configuration of the machine learning apparatus 1 shown in any of FIG. 12 to FIG. 14 may be applied to the present embodiment. In addition, when the training image IT and the correct answer image IC are generated based on the candidate correct answer image ICC instead of the original image, the configuration of the machine learning apparatus 1 shown in FIG. 10 may be applied to the present embodiment.

The endoscope system 41 includes an endoscope 42 and an endoscope image processing apparatus 44. An image pickup apparatus 43 is provided in the endoscope 42, for example. However, the image pickup apparatus 43 may be provided in a camera head, and the camera head may be attached to an eyepiece portion of the endoscope 42. The image pickup apparatus 43 is connected to the endoscope image processing apparatus 44. The endoscope 42, the image pickup apparatus 43, and the endoscope image processing apparatus 44 are hardware.

The image pickup apparatus 43 roughly has a configuration similar to that of the image pickup apparatus 15 shown in FIG. 9, except that the optical low-pass filter 17 is removed, as with the image pickup apparatus 33 shown in FIG. 15.

The image pickup apparatus 43 forms an image of the subject light flux by the image pickup optical system 16, picks up an image by the image pickup device 18, and outputs an endoscopic image. The endoscopic image outputted from the image pickup apparatus 43 is the input image I1 (see FIG. 1) to the endoscope image processing apparatus 44.

The endoscope image processing apparatus 44 includes, for example, a processor 44a and a memory 44b. The processor 44a and the memory 44b are hardware. The processor 44a is configured by an ASIC including a CPU, etc., an FPGA, or the like. However, the endoscope image processing apparatus 44 may be configured as a dedicated electronic circuit that performs a function of the learned model M.

The memory 44b is a storage medium that saves (stores in a non-volatile manner) a processing program that causes the processor 44a to realize a function of the endoscope image processing apparatus 44. The processor 44a is connected to a wiring led out from the memory 44b. The processor 44a reads and executes the processing program stored in the memory 44b to realize the function of the endoscope image processing apparatus 44. For example, the endoscope image processing apparatus 44 realizes an endoscope image processing method by executing the processing program.

A combination of the learned model M, that is, an AI program (algorithm), generated by the machine learning apparatus 1, and parameters optimized by learning is stored in the memory 44b of the endoscope image processing apparatus 44. The memory 44b and the wiring led out from the memory 44b constitute a machine learning model connection section configured to be connectable to the learned model M.

The processor 44a performs the endoscope image processing method to perform inference for the input image I1 by the learned model M, and outputs the inferred image 12 (see FIG. 1). As a result of an appropriate inference performed, the inferred image 12 is an endoscopic image with an improved resolving power compared to the input image I1.

According to the ninth embodiment, the learned model M that has performed learning in the configuration of any of the third to seventh embodiments is applied to the endoscope system 41 to perform an appropriate inference with few faults, thereby obtaining an output image with an improved resolving power.

Note that the above description is mainly made for a case where the present disclosure relates to, but is not limited to, the image generation method for machine learning, the machine learning method, the endoscope image processing method, the image generation apparatus for machine learning, the machine learning apparatus, and the endoscope image processing apparatus. For example, the present disclosure may relate to a computer program that causes a computer to perform processing in accordance with the image generation method for machine learning, the machine learning method, and the endoscope image processing method. Furthermore, the present disclosure relates to a non-transitory computer-readable recording medium that records the computer program, or the like.

Here, some examples of recording media that store computer program products are portable recording media such as a flexible disk, a compact disc read only memory (CD-ROM), and a digital versatile disc (DVD), recording media such as a hard disk drive (HDD), and a solid state drive (SSD), etc. It is not necessary for the entire computer program to be stored in the recording medium, and it is also possible to store only part of the computer program. In addition, the entire or part of the computer program may be distributed or provided via a communication network. A user installs the computer program from the recording medium on a computer, or downloads the computer program via the communication network and installs the computer program on the computer, and the computer reads the computer program and executes the entire or part of operations. The above-described computer can then perform the processing in accordance with the image generation method for machine learning, the machine learning method, and the endoscope image processing method.

Furthermore, the present invention is not limited to the above-described embodiments as they are. The present invention can be embodied by modifying the constituent elements within the scope of not deviating from the gist of the invention at the stage of implementation. In addition, various aspects of the invention can be formed by combining the plurality of constituent elements disclosed in the above embodiments as appropriate. For example, some of the constituent elements may be deleted from all the constituent elements disclosed in the embodiments. Furthermore, the constituent elements in different embodiments may be combined as appropriate. As such, it goes without saying that various modifications and applications are possible within the scope of not deviating from the gist of the invention.

What is claimed is:

1. An image generation method for machine learning, comprising:
for a candidate correct answer image with a resolution higher than a resolution of a training image, the training image being a pair with a correct answer image,
performing reduction processing on components in at least part of a frequency band higher than a Nyquist frequency of the training image; and
generating the correct answer image for machine learning to improve a resolving power of an input image.

2. The image generation method for machine learning according to claim 1, wherein the reduction processing reduces components in overall the frequency band higher than the Nyquist frequency.

3. The image generation method for machine learning according to claim 1, wherein the reduction processing makes components in overall the frequency band higher than the Nyquist frequency 0.

4. The image generation method for machine learning according to claim 1, wherein the reduction processing downscales the candidate correct answer image so that a resolution is lower than the resolution of the candidate correct answer image and equal to or higher than the resolution of the training image, without reducing components of the candidate correct answer image in a frequency band equal to or lower than the Nyquist frequency of the training image.

5. The image generation method for machine learning according to claim 4, wherein the reduction processing downscales the candidate correct answer image so that a resolution is a same as the resolution of the training image.

6. The image generation method for machine learning according to claim 1, wherein the reduction processing further reduces components in at least part of a frequency band equal to or higher than ½ of the Nyquist frequency and equal to or lower than the Nyquist frequency.

7. The image generation method for machine learning according to claim 1, wherein the reduction processing is performed by applying low-pass filter processing to the candidate correct answer image.

8. The image generation method for machine learning according to claim 1, wherein the reduction processing is performed by applying low-pass filter processing to the candidate correct answer image, and downscaling the candidate correct answer image to which the low-pass filter processing has been applied.

9. A machine learning method, comprising:
causing a machine learning model for performing inference to improve a resolving power of an input image and generating an output image to perform learning, using:
a correct answer image that is generated by performing reduction processing on components in at least part of a frequency band higher than a Nyquist frequency of a training image, for a candidate correct answer image with a resolution higher than a resolution of the training image, the training image being a pair with the correct answer image, or
the correct answer image that is picked up by an image pickup apparatus mounting an optical low-pass filter, the optical low-pass filter being configured to reduce components in at least part of the frequency band higher than the Nyquist frequency of the training image; and
the training image.

10. The machine learning method according to claim 9, wherein when the reduction processing is performed, the reduction processing reduces components in overall the frequency band higher than the Nyquist frequency.

11. The machine learning method according to claim 9, wherein when the reduction processing is performed, the reduction processing makes components in overall the frequency band higher than the Nyquist frequency 0.

12. The machine learning method according to claim 9, wherein when the reduction processing is performed, the reduction processing downscales the candidate correct answer image so that a resolution is lower than the resolution of the candidate correct answer image and equal to or higher than the resolution of the training image, without reducing components of the candidate correct answer image in a frequency band equal to or lower than the Nyquist frequency of the training image.

13. An endoscope image processing apparatus, comprising:
a machine learning model connection section that is configured to be connectable to a machine learning model that has performed learning by a machine learning method, the machine learning method causing the machine learning model to perform learning, using:
a correct answer image that is generated by performing reduction processing on components in at least part of a frequency band higher than a Nyquist frequency of a training image, for a candidate correct answer image with a resolution higher than a resolution of the training image, the training image being a pair with the correct answer image, or
the correct answer image that is picked up by an image pickup apparatus mounting an optical low-pass filter, the optical low-pass filter being configured to reduce components in at least part of the frequency band higher than the Nyquist frequency of the training image; and
the training image; and
one or more processors, wherein
the one or more processors are configured to:
input an endoscopic image, which is received, into the machine learning model configured to perform inference to improve a resolving power of an input image and generate an output image; and
cause the machine learning model to output the endoscopic image with a resolving power that is improved.

14. The endoscope image processing apparatus according to claim 13, wherein the reduction processing reduces components in overall the frequency band higher than the Nyquist frequency.

15. The endoscope image processing apparatus according to claim 13, wherein the reduction processing makes components in overall the frequency band higher than the Nyquist frequency 0.

16. The endoscope image processing apparatus according to claim 13, wherein the reduction processing downscales the candidate correct answer image so that a resolution is lower than the resolution of the candidate correct answer image and equal to or higher than the resolution of the training image, without reducing components of the candidate correct answer image in a frequency band equal to or lower than the Nyquist frequency of the training image.

17. The endoscope image processing apparatus according to claim 13, wherein
the machine learning model connection section includes:
a storage medium configured to save the machine learning model; and
a wiring that is led out from the storage medium.
